(19) United States
(12) Patent Application Publication
(10) Pub. No.: US 2005/0209524 A1
(43) Pub. Date: Sep. 22, 2005

(54) System and method for receiving and storing information pertaining to a patient

(75) Inventors: Brenda L. Donaldson, Harrison Township, MI (US); Curtis G. Neason, New York, NY (US)

Correspondence Address: GE MEDICAL SYSTEM;C/O FOLEY & LARDNER, 777 EAST WISCONSIN AVENUE, MILWAUKEE, WI, 53202-5367, US

(73) Assignee: General Electric Company

(21) Appl. No.: 10/797,873

(22) Filed: Mar. 10, 2004

(51) Int. Cl. A61B 5/0402

(52) U.S. Cl. 600/510

(57) ABSTRACT

The subject matter disclosed herein relates to a system which comprises one or more probes configured to be positioned inside a heart of a patient and computer components communicatively coupled together and communicatively coupled to the one or more probes. The computer components are configured to store position information pertaining to a position of at least one of the probes inside the heart. The computer components are also configured to store electrical information sensed using the one or more of the probes. The position information and electrical information are stored in a database and the position information and electrical information are used to create a report.

### BACKGROUND

EP studies can be used to diagnose and treat a number of serious heart problems. One type of heart problem that can be diagnosed and treated by conducting an EP study is a cardiac arrhythmia. A cardiac arrhythmia can generally be referred to as an abnormal heart rhythm such as tachycardia, bradycardia, etc. One particularly dangerous arrhythmia that is often diagnosed and treated using an EP study is ventricular fibrillation. Left untreated, an arrhythmia presents a serious health risk to an individual.

Various types of systems may be used to perform an EP study such as an EP mapping procedure or an EP monitoring and diagnostic procedure. For example, an EP mapping system is used to perform an EP mapping procedure. In an EP mapping procedure, a catheter is inserted into a vein or artery (e.g., in the groin, etc.) and guided to the interior of the heart. Once inside the heart, the catheter is contacted with the endocardium at multiple locations. At each location, the position of the catheter and optionally the activation time can be measured. This information may then be used to create a structural map of the heart showing the activation times. The attending physician uses this information to assist in locating the origin of a cardiac arrhythmia. Generally, once the origin of the arrhythmia is located, the area is ablated using the catheter.

In other instances, an EP monitoring system may be used to acquire more information pertaining to the heart. For example, EP monitoring systems may be used to pace the heart and record the resulting electrical activity. The EP monitoring system may be configured to record 128 channels and include 224

catheter inputs.

Traditionally, the EP mapping system has not been configured to communicate or interface with an EP monitoring system. Therefore, even though both systems may be used as part of an EP study, the information obtained is not organized and stored in a uniform fashion. This makes it difficult and time consuming to create reports that utilize information from both systems. The reports may be used to analyze the procedure for research purposes or as part of follow-up care.

Of course, the claims define the scope of the subject matter for which protection is sought, regardless of whether any of the aforementioned disadvantages are overcome by the subject matter recited in the claims. Also, the terms recited in the claims should be given their ordinary and customary meaning as would be recognized by those of skill in the art, except, to the extent a term is used herein in a manner more expansive than its ordinary and customary meaning, the term should be given its ordinary and customary meaning plus the additional expansive meaning, or except if a term has been explicitly defined to have a different meaning by reciting the term followed by the phase “as used herein shall mean” or similar language. Accordingly, the claims are not tied to any particular embodiment, feature, or combination of features other than those explicitly recited in the claims.

### SUMMARY

According to one embodiment, a system comprises one or more probes configured to be positioned inside a heart of a patient and computer components communicatively coupled together and communicatively coupled to the one or more probes. The computer components are configured to store position information pertaining to a position of at least one of the probes inside the heart. The computer components are also configured to store electrical information sensed using the one or more of the probes. The position information and electrical information are stored in a database and the position information and electrical information are used to create a report.

According to another embodiment, a system comprises an electrophysiological monitoring system which includes electrical information pertaining to a heart, an electrophysiological mapping system which includes position information that is used to create a structural map of the heart, and a database which is used to store the electrical information and the position information. The electrical information and the position information in the database are correlated to each other. According to another embodiment, a system comprises one or more probes configured to be positioned inside a heart of a patient, at least one of the probes being used to pace the heart. The system also comprises computer components communicatively coupled together and communicatively coupled to the one or more probes. The computer components are configured to log electrical information sensed using the one or more probes while the heart is being paced. The computer components are also configured to store position information pertaining to a position of at least one of the probes inside the heart. The electrical information and the position information are correlated to each other.

According to another embodiment a method comprises periodically storing electrical information sensed using one or more probes positioned inside a heart of a patient and storing position information pertaining to a position of one or more probes positioned inside a heart of a patient. The electrical information and the position information is stored in a database. The method also comprising generating a report using the electrical information and position information from the database.

### BRIEF DESCRIPTION OF THE DRAWINGS

FIG. 1 is a system for receiving information pertaining to a patient according to one embodiment.

FIG. 2 is a display for receiving and storing information pertaining to a patient in a database according to another embodiment.

FIG. 3 is a flowchart showing one embodiment of a method for receiving and/or storing information pertaining to a patient.

### DETAILED DESCRIPTION

The subject matter described herein is generally referred to in the context of a system which is configured to receive and/or store information pertaining to the heart of a patient (e.g., electrical information pertaining to the heart, structural information pertaining to the heart, etc.). Although, the present description is provided primarily in the context of receiving, storing, and using electrical information and structural information, it should be understood that the systems and methods described and claimed herein may also be used in other contexts as would be recognized by those of ordinary skill. It should also be understood that a particular example or embodiment described herein may be combined with one or more other examples or embodiments also described herein to form various additional embodiments as would be recognized by those of ordinary skill. Also, reference to various features in singular tense should also be understood as equally including the plural tense unless noted otherwise. Accordingly, the systems and methods described herein may encompass various embodiments and permutations as may be desired.

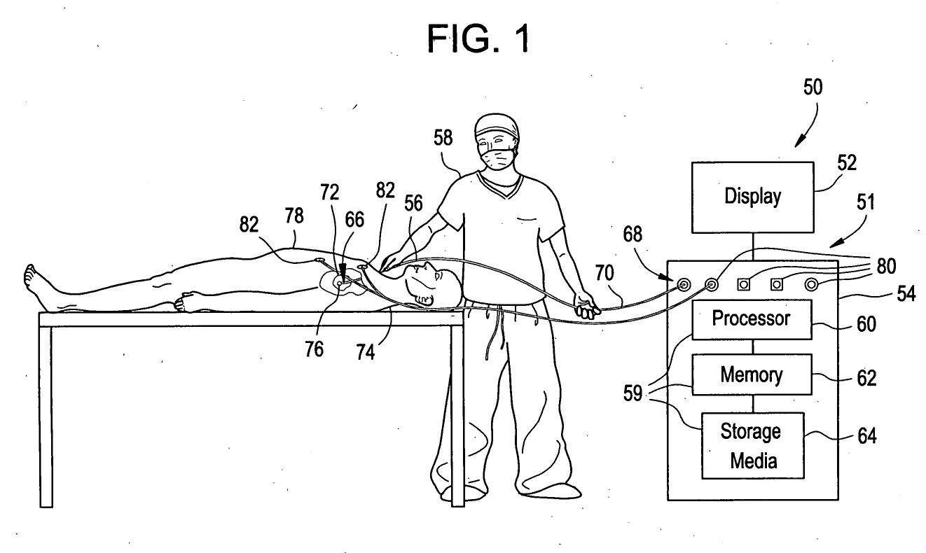

Referring to FIG. 1, one embodiment of a system 50 is shown. System 50 includes a console or computer 51 and a probe 56. System 50, broadly described, may be used to receive, store, and display various types of information. In particular, system 50 may be used to simultaneously and/or selectively receive, store, and/or display electrical and/or structural information pertaining to a heart 72 of a patient 74.

In general, system 50 may be any system that is configured to use one or more probes 56 positioned inside the body to measure, monitor, diagnose, manipulate, and/or otherwise provide information about heart 72. In particular, system 50 may be used to create a structural map of heart 72 as well as to pace heart 72 and record electrical information pertaining to heart 72.

As shown in FIG. 1, probe 56 and display 52 are communicatively coupled to computer components 59 in cabinet 54. Information sensed by probe 56 may be communicated to computer components 59. Information from computer components 59 may then be communicated to display 52 where it is displayed to a nearby person 58 (e.g., attending physician, nurse, technician, etc.). The configuration shown in FIG. 1 is only one of many suitable configurations. For example, in another embodiment, probe 56 may be communicatively coupled directly to display 52. In this embodiment, display 52 may be configured to display the information provided by probe 56 without the information being communicated through cabinet 54 (e.g., display 52 comprises the computer components 59 which are used to receive information from probe 56). In another embodiment, display 52 may be combined with cabinet 54 so that the functions generally performed by computer components 59 in cabinet 54 and display 52 are performed by the combined unit (e.g., display 52 comprises all of computer components 59). In another embodiment, console 51 may include two or more displays 52. For example, one display may be used to display electrical and/or structural information pertaining to heart 72 and the other may be used to display an image such as a computed tomography (CT), magnetic resonance MR, or ultrasound image. Of course, a wide variety of information may be displayed on display 52. In one embodiment, display 52 may be configured to be at a position that is convenient for person 58 to view (e.g., display 52 is positioned at eye level of person 58 when person 58 is standing, etc.) as person 58 moves probe 56.

System 50 may be configured to include additional components and systems. For example, system 50 may comprise a printer. The printer may be configured to print on standard sized paper or may be configured to print on smaller rolls of paper. The printer may also be used to print out a report at the end of an EP study. System 50 may also be configured as part of a network of computers (e.g., wireless, cabled, secure network, etc.) or as a stand-alone system. Information pertaining to patient 74 may be transmitted over the network and stored as part of a data record for patient 74.

In one embodiment, system 50 may be configured to receive an X-ray image (e.g., fluoroscopy image) and correlate the image to the electrical information sensed using probe 56. Typically, this is done by gating and correlating (e.g., temporally, reference tags, etc.) the image data and the electrical information as they are received by system 50. System 50 may be used to view both live or real-time fluoroscopy images and stored images. The images may be displayed alone or simultaneously (e.g., a live image and a stored image are displayed). The images may be annotated with comments or to mark areas of interest (e.g., activation sites, etc.).

In another embodiment, system 50 maybe provided as part of a network. For example, the network may be a health care facility network (e.g., hospital, clinic, etc.), the Internet, wide area network, etc. In this configuration, users from remote locations on the network are able to access and manipulate the information acquired using system 50. Users on the network may be able to join an EP study as it is happening. Thus, multiple users can see the results of the EP study without being in the room where it is happening. Also, a camera (e.g., video camera, periodic still camera, etc.) and a microphone may be used to acquire video and audio signals and transmit those to the users over the network. Thus, the users on the network are able to observe more closely and feel more involved in the procedure without being an extra body in operating room.

In another embodiment, system 50 is configured to allow a user to enter notes about the patient/procedure. Also, system 50 may be configured to log the details of a particular EP study (e.g., medication information such as type, amount, times administered, etc., pacing information, ablations information, etc.). This data is typically logged at regular intervals throughout the procedure. This data can be used during the procedure or after the procedure to further analyze and diagnose the problem. This may be useful when the problem continues even after the procedure is over.

In another embodiment, system 50 may be configured to include a number of individual commands. These individual commands may be combined in a single command using manufacturer configured or user configured macros. The macros allow the user to customize system 50 in any desirable manner. For example, a macro may be created which is used to end a case. When this macro is activated, system 50 is commanded to take a final vitals measurement, print a report, and stop recording electrical information. Thus, the user does not need to perform each of these commands separately each time a procedure is completed.

In another embodiment, system 50 may be configured to use a Holter window. Also, system 50 may be configured to include an alignment window, which allows the user to compare signals from different locations to each other.

System 50 may be configured to receive, store, and/or display various information pertaining to patient 74. For example, in one embodiment, system 50 may be configured to receive, store, and/or display vitals information pertaining to patient 74. Vitals information may include one or more, in any combination, of the following types of patient information: pulse oximetry (SpO₂), non-invasive blood pressure NP), temperature, respiratory rate, respiratory CO₂ concentration (etCO₂), impedance cardiography (ICG), pulse rate, cardiac output (CO), etc. System 50 may also include sensors that are communicatively coupled to computer components 59 in console 51 to provide this information. In one embodiment, display 52 may be configured to display at least one, two, three, four, or all five of the following types of information pertaining to patient 74: non-invasive blood pressure, temperature, respiratory rate, pulse oximetry, respiratory CO₂ concentration, and pulse rate.

Computer components 59 in cabinet 54, shown in FIG. 1, comprise a processor 60, memory 62, storage media 64, and one or more input devices (e.g., mouse, keyboard, etc.). Computer components 59 are configured to receive information from probe 56, process the information, and provide output using display 52. The information provided to computer components 59 may be continually stored (i.e., all information is stored as it is received) or intermittently stored (i.e., periodic samples of the information are stored or logged) using storage media 64 (e.g., optical storage disk such as a CD, DVD, etc., high performance magneto optical disk, magnetic disk, etc.). In general, storage media 64 differs from memory 62 in that storage media 64 is configured to maintain the information even when storage media 64 is not provided with power. In contrast, memory 62 typically does not maintain the information when the power is off.

In one embodiment, console 51 is a desktop computer. In another embodiment, console 51 may include input receivers 80 on cabinet 54 or display 52 that are configured to receive additional information pertaining to patient 74. For example, in one embodiment, input receivers 80 may include one or more input receivers configured to receive input from leads 82 (e.g., ECG leads, etc.). In other embodiments, input receivers 80 may include suitable receivers for receiving vitals information. For example, input receivers 80 may be configured to be coupled to a traditional NIBP arm cuff sensor.

Probe 56 comprises a distal end 66, a proximal end 68, and a probe body 70. In general, probe 56 may be positioned in or adjacent to heart 72 (shown in FIG. 1 in a cross-sectional view to expose distal end 66 of probe 56) of patient 74. In one embodiment, distal end 66 may include one or more sensors 76, which are configured to sense various electrical information (e.g., electrical potential at one or more positions of the endocardium, activation times, etc.) pertaining to heart 72. The electrical information may then be communicated back to console 51 and displayed on display 52 or stored on storage media 64. In one embodiment, probe 56 may comprise a plurality of sensors configured to sense the electrical information pertaining to heart 72 (e.g., probe 56 is a balloon or sock catheter, etc.). The electrical information may be used to create an electrical map (e.g., map of the activation times, electrical potentials, etc.) of heart 72.

Probe 56 may be any number of suitable probes having a variety of configurations. For example, probe 56 may include a lumen in which wires may be placed to communicate information from sensors 76 back to console 51 and to transmit an ablation charge from console 51 to distal end 66 to correct the electrical pathways in heart 72. Of course, the lumen may also be used to allow fluid to flow through probe 56.

In another embodiment, a localization system, included as part of system 50, may be used to determine the spatial location of one or more portions (e.g., sensors 76, etc.) of distal end 66 of probe 56. This may be useful in moving probe 56 back to an earlier position or to create a structural map of heart 72. Any suitable localization system may be used as would be recognized by those of ordinary skill. For example, the position of distal end 66 of probe 56 may be determined using one or more transmitters and/or receivers that are located outside the body of patient 74 (typically at least three transmitters and/or receivers are used). In this example, the transmitters and/or receivers may be configured to send and/or receive signals to and/or from distal end 66. These signals may be used to determine the position of distal end 66. In one embodiment, the transmitters and/or receivers may be incorporated into one or more leads 82 positioned on skin surface 78 of patient 74. In another embodiment, the transmitters and/or receivers may be positioned so as not to be in contact with patient 74. In another embodiment, leads 82 may be used to determine the position of distal end 66 of probe 56 by sending a signal that is useful in determining the impedance of probe 56, which may be used to determine the position of probe 56. In another embodiment, the localization system may be configured to determine the position of multiple sensors 76 on distal end 66 of probe 56. In another embodiment, the position of probe 56 may be determined using a magnetic field.

Display 52, shown in FIG. 1, is configured to provide output to a user such as alphanumeric (e.g., text, numbers, etc.) output, graphical image output, etc. In one embodiment, display 52 may be configured to also receive input from a user (e.g., touch screen, buttons located adjacent to the screen portion of display 52, etc.). Display 52 may be any number of suitable displays in any number of suitable configurations. For example, display 52 may be a liquid crystal display, flat screen display, SVGA display, VGA display, etc.

In one embodiment, display 52 may be configured to display one or more images (CT, MR, ultrasound, etc.) of heart 72. Display 52 may also be configured to display a structural and/or electrical map of heart 72. In another embodiment, display 52 may be configured to display vitals information pertaining to patient 74.

Display 52 may also be configured to display one or more representations of one or more probes 56 and the information provided by probes 56. For example, in one embodiment, display 52 may be configured to display a representation of probe 56. In another embodiment, display 52 may be configured to display representations of sensors 76 which are on probe 56. In another embodiment, display 52 may be configured to display the electrical information pertaining to heart 72, which is received from sensors 76 (e.g., a contour map of the electrical properties of heart 72). In another embodiment, display 52 may be configured to display markers showing one or more locations where the electrical information has been sensed. In one embodiment, each marker may display an abbreviated amount of information regarding the electrical information. When a user selects one of the markers, the user is shown a greater amount of electrical information for that particular location of heart 72. The markers may be color coded based on the activation times at the various locations inside heart 72 (e.g., red is for early activation times and blue is for late activation times). By displaying a number of markers on display 52, the user can readily observe the electrical information pertaining to various areas of heart 72. Any suitable marker or identifier may be used to represent probe 56 on display 52. For example, in one embodiment, probe 56 may be displayed as a line with a series of points corresponding to sensors 76. The line segments connecting the points represent the portion of probe 56 where there are no sensors. Probe 56 may be shown or represented on display 52 in any of a number of other suitable ways as well.

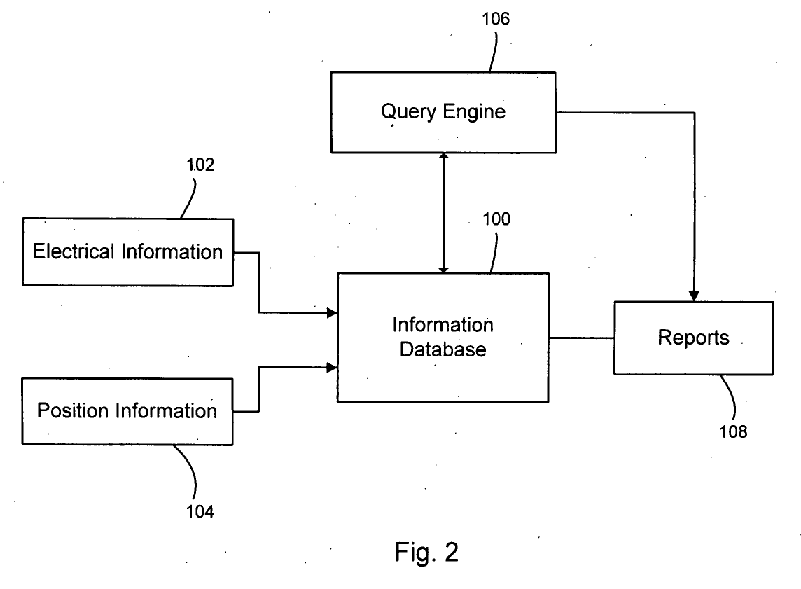

Referring to FIG. 2, one embodiment of system 50 is shown for storing electrical information 102 and position information 104 received from probe 56 in a database 100. In general, database 100 may comprise patient information which is information that is unique to a particular patient. Database 100 may be any suitable database to provide the desired capabilities. Typically, however, database 100 is a file consisting of a number of records or tables each of which is constructed of fields of a particular type, together with a collection of operations that facilitate searching, sorting, recombination, and/or similar activities. In one embodiment, database 100 is a relational database which is used to correlate electrical information 102 and position information 104 to each other. In another embodiment, database 100 may be a flat file database. As mentioned previously, electrical information 102 and position information 104 may be correlated temporally or in some other suitable manner (e.g., identification tags, etc.). Database 100 typically comprises a database management system which is a layer of software between the user and the collection of data. The database management system manages all request for database action (e.g., queries, updates, etc.) from the user. Thus, the user is spared the tasks of keeping track of the physical details of file locations and formats, indexing schemes, and so on.

Database 100 comprises a number of fields that are filled or populated as the information is received. In addition to electrical information 102 and position information 104, database 100 may be used to store a wide variety of additional information such as vitals information, patient history, procedure type, medications used, etc. In one embodiment, database 100 may be hosted on a network so that it is accessible to multiple systems on the network. Thus, system 50 may be configured to record or store information to database 100 regardless of its location. Also, system 50 may be able to retrieve information from database 100 across a network.

Query engine 106 is provided to allow users to search the various information stored in database 100. In one embodiment, query engine 106 may be used to query database 100 during an EP study. In other embodiments, query engine may be used to query database 100 after an EP study for research or other purposes. In one embodiment, the information may be queried using any combination of information stored in database 100. Also, information about multiple patients may be queried as well. For example, a query may be used to locate information about patients studied in a two year window with atrial fibrillation and ERP>300. In another example, a query may be used to find correlations between electrical information, position information, and patient information. For example, a physician may be able to perform a query to determine if a voltage of 0.1 mV across the apex of the right ventricle correlates with a prolonged QRS duration, and an inferior MI which was not acutely treated.

The information stored in database 100 may be used to create a number of reports 108. For example, a report may be created at the end of each EP study summarizing the information acquired during the study, the treatment methods (e.g., RF ablation, medication, etc.). The report may also include electrical and structural maps of heart 72. By including detailed electrical information about heart 72, such as ECG readings during pacing, and a structural map or image that is correlated, and in some instances registered with the electrical information, the attending physician has a simple and easy reference for the future. It may be useful for the physician to refer to reports 108 for research purposes or, if the problem occurs again, to determine likely causes for the recurrence.

In one embodiment, reports 108 comprise a number of fields that correspond to the fields in database 100. Thus, reports 108 may be easily created by populating the fields in reports 108 with corresponding information from database 100. This may be combined with the macro feature to provide an efficient way to prepare a report in conjunction with other commands (“end of case” command described above).

In one embodiment, reports 108 may comprise vitals information as well as additional information such as the name of the physician performing the EP procedure, the name of the nurse that is present, medications patient 74 may be taking, allergies, patient history, and/or a description of the procedure. The description of the procedure may provide information about probe 56 (e.g., type of probe, location where probe 56 is inserted into the body, etc.). Reports 108 may also include electrical information pertaining to heart 72. For example, reports 108 may include information resulting from pacing heart 72 (e.g., site where pacing was induced, etc.) and/or information about any induced arrhythmias and, in particular, ventricular tachycardia. Reports 108 may also include information pertaining to a structural map of heart 72 such as a structural map of heart 72 or information pertaining to the location of probe 56 as it is moved around inside heart 72. Reports 108 may also include information pertaining to treatments performed during the procedure. For example, reports 108 may include information about the location and time of an ablation. All of this information may be provided to the physician in an easy to read and understand manner. Reports 108 may be especially useful later when examining the patient's 74 medical history to determine any problems or history of illness associated with patient 74.

The following is one embodiment of a report 108 that may be generated using the information from database 100. Also, information may be entered manually into the report without being entered into database 100. The information provided in following report 108 is only meant to show various types of information that may be used in a particular field, cell, or location and is not meant to represent actual data obtained from a patient. Also, it should be understood that much of the information included in the following report 108 may be provided by populating a field in report 108 with corresponding information from database 100.

| | |
|---|---|
| Referring Physician: | Referring Physician, MD |
| Primary Care Physician: | Attending Physician, MD |
| Nurse: | Attending Nurse |
| Tech: | Technician |
| Current Medications: | None |
| Allergies: | None |

History: 40 year old male with Hepatitis C and ex-IV drug abuse with known WPW since age 17. He has had infrequent palpitations in the past but recently had an episode of prolonged palpitations and was evaluated for ablation.

Procedure:

After informed written consent was obtained the patient was transported to the electrophysiology laboratory in the post absorptive, non-sedated state. The patient was prepped and draped in the usual sterile manner. A 1% Lidocaine solution was used for local anesthesia. A combination of Fentanyl, Droperidol and Morphine were used for conscious sedation throughout the procedure. The patient was continuously monitored throughout the case per hospital standards. The following sheaths were placed, after local anesthesia, using the Seldinger technique. In addition the following electrode catheters were placed under fluoroscopic guidance.

| | | | Cath- | | | Cath- | |
|---|---|---|---|---|---|---|---|
| Site | Sheath | | eter | Location | Location | eter | Location |
| 1 | 5 | F Cordis | | HRA | HRA | | HRA |
| 2 | 6 | F Cordis | | RVA | RVA | | RVA |
| 3 | 7 | F Cordis | | RVOT | RVOT | | RVOT |
| 4 | 8 | F Cordis | | CS | CS | | CS |
| 5 | 6.5 | F Locking | | RA | RA | | RA |
| 6 | 10 | F Duo | | Tricupid | Tricupid | | Tricupid |
| | | | | Ann | Ann | | Ann |
| 7 | 11 | F Duo | | LA | LA | | LA |
| 8 | 11 | F Trio | | LV | LV | | LV |

After baseline conduction intervals were recorded, programmed extra-stimulation was performed. Atrial overdrive pacing and extra-stimulation was performed from the HRA. Ventricular overdrive pacing and extra-stimulation was performed with up to three extra-stimuli from the LV. Following intravenous administration of Procainimide programmed stimulation was repeated.

At the end of the procedure the catheters and sheaths were removed and hemostasis was achieved with pressure. The patient was transported back to the recovery room in good condition.

Results:

At baseline the patient was in atrial fibrillation with a CL of ______.

Baseline conduction intervals were:

PR______ QRS______ QT______ AH______ HV______

Ventricular Pre-excitation was present.

Corrected sinus node recovery time was ______.

AV Nodal Conduction

AV Nodal block cycle length ______.

AV Nodal VA block cycle length ______.

VA conduction was not Decremental.

| Pacing Site | Refractory Site | Drive | ERP |
|---|---|---|---|
| HRA | Atrium | | |
| RVA | AV Node | | |
| RVOT | Ventricle | | |
| CS | Atrium | | |
| LA | AV Node | | |
| LV | Ventricle | | |

Results Post Procainamide Infusion:

The rhythm was SVT with a cycle length of ______.

Conduction intervals were: PR______ QRS______ QT______ AH______ HV______

Ventricular Pre-excitation was not present.

Corrected sinus node recovery time was ______.

AV Nodal Conduction

AV Nodal block cycle length ______.

AV Nodal VA block cycle length ______.

VA conduction was Decremental.

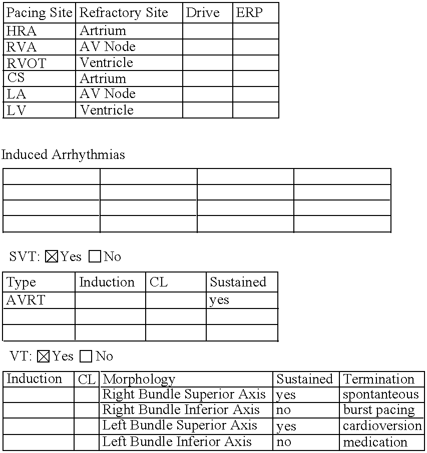

Mapping:

After the baseline study was completed extensive endocardial mapping was performed.

[Image showing a map of the heart would be inserted here]

Ablation:

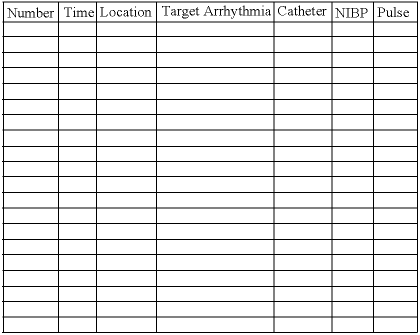

Post Ablation Extra-Stimulation:

The rhythm was sinus rhythm with a cycle length of ______.

Conduction intervals were: PR______ QRS______ QT______ AH______ HV______

Ventricular Pre-excitation was present.

Corrected sinus node recovery time was ______.

AV Nodal Conduction

AV Nodal block cycle length ______.

AV Nodal VA block cycle length ______.

VA conduction was not Decremental.

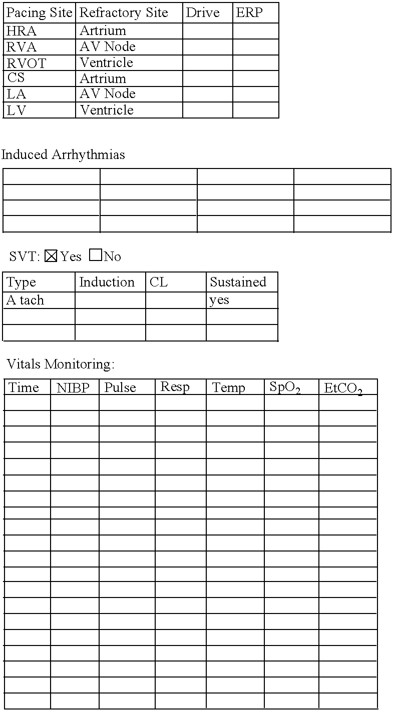

Findings:

1.

2.

3.

Plan:

1.

2.

3.

______, M.D.

Director of Electrophysiology Laboratory

The report shown above is only one example of a suitable report. Accordingly, numerous alterations may be made to the format of the information and what information is included. For example, in one embodiment, the report may include vitals information such as non-invasive blood pressure, temperature, respiratory rate, pulse oximetry, respiratory CO₂ concentration, and pulse rate. In another embodiment, the report may include graphs of various vitals information recorded during the procedure (e.g., graph of blood pressure, pulse, etc.). In another embodiment, the report may include an electrical map of heart 72. In another embodiment, the report may include a structural map of the structure of heart 72 acquired by measuring multiple locations of probe 56.

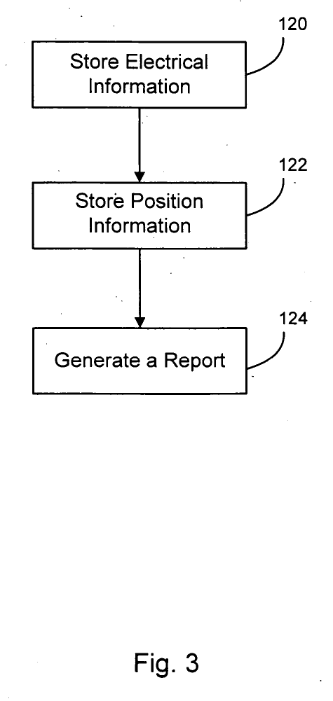

Referring to FIG. 3, a method is shown for creating or generating a report 108 using system 50. At step 120, electrical information 102 pertaining to heart 72 is received by computer components 59 of system 50. At step 122, position information 104 pertaining to the position of one or more probes 56 in heart 72 is received by system 50. Although steps 120 and 122 are shown as being sequential in FIG. 3, it should be understood that electrical information 102 and position information 104 may be received simultaneously or at various times throughout an EP study. Thus, electrical information 102 and position information 104 may be received in any suitable manner. In one embodiment, electrical information 102 and position information 104 are stored in database 100 as explained previously.

At step 124 report 108 is generated using electrical information 102 and position information 104. In one embodiment, as described previously, report 108 may comprise a plurality of fields which are populated with electrical information 102 and position information 104 from corresponding fields in database 100.

The construction and arrangement of the elements described herein are illustrative only. Although only a few embodiments have been described in detail in this disclosure, those of ordinary skill who review this disclosure will readily appreciate that many modifications are possible without departing from the spirit of the subject matter disclosed herein. Accordingly, all such modifications are intended to be included within the scope of the methods and systems described herein. The order or sequence of any process or method steps may be varied or re-sequenced according to alternative embodiments. Other substitutions, modifications, changes and omissions may be made in the design, operating conditions and arrangement of the embodiments without departing from the spirit and scope of the methods and systems described herein.

1. A system comprising:
one or more probes configured to be positioned inside a heart of a patient;
computer components communicatively coupled together and communicatively coupled to the one or more probes, the computer components being configured to store position information pertaining to a position of at least one of the probes inside the heart, the computer components also being configured to store electrical information sensed using the one or more of the probes;
wherein the position information and electrical information are stored in a database and the position information and electrical information are used to create a report.

2. The system of claim 1, wherein the electrical information comprises electrical information obtained while pacing the heart.

3. The system of claim 1, wherein the report comprises a structural map of the heart.

4. The system of claim 1, wherein the position of at least one of the probes is determined using a magnetic field.

5. The system of claim 1, wherein the electrical information and position information are stored in a plurality of fields in the database, and wherein the report includes a plurality of fields which are populated using the electrical information and position information from the corresponding plurality of fields in the database.

6. The system of claim 1, further comprising a single console which comprises the computer components.

7. The system of claim 1, wherein the database comprises position information and electrical information from a plurality of patients.

8. The system of claim 1, wherein the report comprises position information and electrical information from a plurality of patients.

9. A system comprising:
an electrophysiological monitoring system which includes electrical information pertaining to a heart;
an electrophysiological mapping system which includes position information that is used to create a structural map of the heart; and
a database which is used to store the electrical information and the position information;
wherein the electrical information and the position information in the database are correlated to each other.

10. The system of claim 9, the electrical information and the position information are used to create a report.

11. The system of claim 10, wherein the electrical information and position information are stored in a plurality of fields in the database, and wherein the report includes a plurality of fields which are populated using the electrical information and position information from the corresponding plurality of fields in the database.

12. The system of claim 10, wherein the report comprises vitals information.

13. The system of claim 9, wherein the electrical information comprises electrical information sensed while the heart is being paced.

14. The system of claim 9, wherein the position information pertains to the position of one or more probes inside the heart.

15. The system of claim 9, wherein the database comprises position information and electrical information from a plurality of patients.

16. The system of claim 9, wherein the report comprises position information and electrical information from a plurality of patients.

17. A system comprising:
one or more probes configured to be positioned inside a heart of a patient, at least one of the probes being used to pace the heart;
computer components communicatively coupled together and communicatively coupled to the one or more probes, the computer components being configured to log electrical information sensed using the one or more probes while the heart is being paced, the computer components also being configured to store position information pertaining to a position of at least one of the probes inside the heart;
wherein the electrical information and the position information are correlated to each other.

18. The system of claim 17, wherein the electrical information and the position information is used to create a report.

19. The system of claim 18, wherein the report comprises vitals information.

20. The system of claim 17, wherein the electrical information and the position information are stored in a database.

21. The system of claim 20, wherein the electrical information and position information from the database are used to generate a report, the report comprising a plurality of fields which are populated with corresponding electrical information and position information from the database.

22. The system of claim 21, wherein the report comprises position information and electrical information from a plurality of patients.

23. The system of claim 20, wherein the database comprises position information and electrical information from a plurality of patients.

24. A method comprising:
storing electrical information sensed using one or more probes positioned inside a heart of a patient, the electrical information being stored in a database;
storing position information pertaining to a position of one or more probes positioned inside a heart of a patient, the position information also being stored in the database;
generating a report using the electrical information and position information from the database.

25. The method of claim 24, wherein the report comprises a structural map of the heart of the patient.

26. The method of claim 24, wherein the report comprises electrical information and position information from a plurality of patients.

27. The method of claim 24, wherein the report comprises pacing information and/or ablation information.

28. The method of claim 24, wherein the database includes a first plurality of fields and the report includes a second plurality of fields, the report being generated by populating the second plurality of fields with the electrical information and position information from the first plurality of fields.

29. The method of claim 24, wherein the report comprises vitals information pertaining to the patient.
